(19) United States
(12) Patent Application Publication
(10) Pub. No.: US 2026/0168973 A1
(43) Pub. Date: Jun. 18, 2026

(54) CONTEXT ENHANCED VAPE DETECTION

(71) Applicant: MOTOROLA SOLUTIONS, INC., CHICAGO, IL (US)

(72) Inventors: JOHN B PLUNKETT, PORT WASHINGTON, NY (US); PIETRO RUSSO, MELROSE, MA (US); PAUL GALBURT, CHICAGO, IL (US); FRANCIS JACOVINO, NORTHPORT, NY (US); SOHAIB SHAIKH, FARMINGTON, CT (US)

(21) Appl. No.: 18/981,994

(22) Filed: Dec. 16, 2024

(51) Int. Cl. G01N 33/00; G01N 15/10; G10L 25/51

(52) U.S. Cl. CPC G01N 33/0027; G01N 15/10; G10L 25/51; G01N 2015/1024

(57) ABSTRACT

A device, system and method for detecting an air pollutant in a location is described. A input sample comprising an air pollutant that is not permitted in a location is received by an air sensor. Audio data that corresponds to the location is received by the air sensor from an audio sensor. A parameter for the type of audio data is set based on a type of the audio data. The detection score of the air sensor is adjusted by a quantity corresponding to the type of audio data. An alert indicative of a presence of the air pollutant in the location is generated by the air sensor based on a comparison of the detection score with a detection threshold.

### BACKGROUND OF THE INVENTION

In some environments, especially in cases of attempted evasion, detection of a substance in the air such as an air pollutant may be challenging. In particular, when an individual, group, or other party engage in a prohibited activity that emits air pollutants, it can be difficult for a sensor to make an accurate detection especially if those engaged in the prohibited activity try to avoid detection. For example, to avoid an accurate detection by the sensor, those who are smoking or vaping may use a masking substance to reduce the sensitivity or confuse the sensor performing vape detection. Improving the ability of the sensor to detect air pollutants may be beneficial for increasing accuracy, reducing false positives, and reducing the time required to make a correct air pollutant detection.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 depicts a block diagram of an example air sensor for air pollutant detection, in accordance with some examples.

FIG. 2 depicts a top view of an example air sensor and corresponding components, in accordance with some examples.

FIG. 3 illustrates a workflow of a method for context enhanced air pollutant detection, in accordance with some examples.

FIG. 4 illustrate a block diagram of an example electronic device, in accordance with some examples.

FIGS. 5A-5B each illustrate an example security ecosystem comprising a plurality of camera devices, in accordance with some examples.

FIG. 6 is a flowchart of a method for improved air pollutant detection based on contextual cues, in accordance with some examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

It may be challenging to provide air pollutant detection, especially when a masking substance that obscures detection of the air pollutant exists in an input sample received by an air sensor. For example, at a school or other educational context, detection of nicotine and/or THC based vaping activity can be difficult to accurately detect especially when the user is attempting to evade detection. In particular, a sensor (e.g., air sensor) configured to sense the presence of vaping activity may experience reductions in sensitivity and/or effectiveness (e.g., can be confused) based on a masking substance that obscures detection of the vaping activity. As an example, the masking substance can be an aerosol spray that masks a vape plume in an input sample received by the air sensor. Consequently, it may be difficult for the air sensor to perform accurate air pollutant detection in some environments and circumstances.

Using additional contextual information or cues may be beneficial based on providing additional information that can improve the accuracy and reduce the number of false instances of detection by the air sensor. Moreover, corroborating air pollutant detection with contextual information (e.g., audio information) can improve the speed of detection (i.e., reducing the amount of time needed for the air sensor to identify the presence of pollutants). Contextual information can be collected or measured by other types of sensors that are in operative communication with the air sensor. As an example, contextual information can be gathered via audio sensors, motion sensors, thermal sensors, and/or the like. For example, various types of contextual information can include audio classification detections (e.g., ignition of a lighter or match, a cough, aerosol spray), audio keyword detections (e.g., phrases such as “give me a hit”), motion detections, thermal detections (e.g., flame from a cigarette lighter). This contextual information may be used to improve the detection result accuracy of the air sensor by adjusting a detection score and/or threshold.

In this way, the air pollutant detection of the present disclosure advantageously may be improved by reducing false positives and missed vape detection or other detection of substances in an air input sample. Accordingly, the present disclosure provides a technical solution to the particular technical challenges of air pollutant described herein. In particular, a detection score and/or threshold (e.g., for detecting a pollutant of interest in the air input sample) can be modified or adjusted based on the contextual information. For example, a detection score such as an aggregate total detection score is compared to a detection score threshold to determine if a positive detection of the pollutant has occurred. The aggregate detection score can be changed (e.g., added to in value) based on sensing certain contextual events have occurred in a timely fashion. That is, contextual clue(s) that are detected as present in the environment of interest can be used as components of the aggregate detection sore in order to improve vape detection.

The present disclosure provides a technical solution to the particular technical challenges of accurate air pollutant (e.g., vape) detection, especially in environments where the source of the air pollutant is disguised, obscured, or masked (either intentionally or unintentionally). Audio analytics and sensor output information constitute contextual cues that are used to improve accurate and quick pollutant detection. In particular, an adder based algorithm including a combination of contextual factors and air pollutant detection may be employed and provide benefits to air pollutant detection. As an example, the contextual factors may include human presence detection, audio cough detection, audio aerosol spray detection, and audio keyword detection. For example, each of the contextual factors can add a specific quantity to a base air pollutant detection score (from the air sensor) to calculate a total detection score.

The specific quantity can be fixed or vary and can be determined/adjusted based on the specific identity of the contextual factor being monitored. The calculated total score may be compared to a detection threshold such that an alert indicative of a presence of the air pollutant in the location is generated if the threshold is exceeded. This algorithm including detection by the air sensor and contextual sensor of the corresponding factors being monitored can be repeated cyclically; that is, a clock timer increment can be applied so that when every given tick of the timer elapses, each of the sensors perform a detection. In this way, the disclosed technical solution advantageously improves air pollutant detection by incorporating and considering contextual factors that function as corroborative/collaborative or confirmatory indicia of the presence of the air pollutant.

According to one embodiment of the present disclosure, a computer-implemented method for detecting an air pollutant in a location is provided. The method includes receiving, by an air sensor, an input sample comprising an air pollutant that is not permitted in a location, wherein the input sample corresponds to a detection score. The method includes receiving, by the air sensor and from an audio sensor, audio data that corresponds to the location. The method includes setting, based on a type of the audio data, a parameter for the type of audio data. The method includes adjusting, based on the parameter, the detection score of the air sensor by a quantity corresponding to the type of audio data. The method includes generating, by the air sensor and based on a comparison of the detection score with a detection threshold, an alert indicative of a presence of the air pollutant in the location.

According to one embodiment of the present disclosure, a system is provided that is configured for detecting an air pollutant in a location. The system includes: an air sensor configured to sense that an air pollutant of interest is present; a motion sensor configured to sense movement at the location; an audio sensor configured to sense audio data audio data that corresponds to the location; and a processor operatively in communication with the air sensor, motion sensor, and the audio sensor, that, upon executing program instructions performs a method for detecting the air pollutant. The air sensor receives an input sample comprising an air pollutant that is not permitted in a location. The processor is configured to determine a detection score for the input sample. The processor is configured to set, based on a type of the audio data, a parameter for the type of audio data. The processor is configured to adjust, based on the parameter, the detection score of the air sensor by a quantity corresponding to the type of audio data. The processor is configured to determine an aggregate detection score based on the adjusted detection score and motion data from the motion sensor. The processor is configured to generate, by the air sensor and based on a comparison of the aggregate detection score with a detection threshold, an alert indicative of a presence of the air pollutant in the location.

According to one embodiment of the present disclosure, an air sensor device including a pollutant sensor, a processor, and a computer-readable storage medium including instructions (e.g., stored sequences of instructions) that, when executed by the processor, cause the air sensor device to perform a method for detecting an air pollutant in a location is provided. The pollutant sensor is configured to sense that an air pollutant of interest is present based on a received input sample comprising an air pollutant that is not permitted in a location. The method includes determining a detection score for the input sample. The method includes receiving, from an audio sensor, audio data that corresponds to the location. The method includes receiving, from a contextual sensor, contextual data corresponding to the location. The method includes setting, based on a type of the audio data, an audio parameter for the type of audio data. The method includes setting, based on a type of the contextual data, a contextual parameter for the type of contextual data. The method includes adjusting, based on the audio parameter and the contextual parameter, a detection score of the air sensor by a quantity corresponding to the type of audio data and the type of contextual data. The method includes generating, based on a comparison of the detection score with a detection threshold, an alert indicative of a presence of the air pollutant in the location.

Each of the above-mentioned embodiments will be discussed in more detail below, starting with example system and device architectures of the system in which the embodiments may be practiced, followed by an illustration of processing blocks for achieving an improved technical communication or data processing based method, device, and system for detecting an air pollutant in a location.

Example embodiments are herein described with reference to flowchart illustrations and/or block diagrams of methods, apparatus (systems) and computer program products according to example embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a special purpose and unique machine, such that the instructions, which execute via processor of the computer or other programmable data processing apparatus, create means for implementing the functions/acts specified in the flowchart and/or block diagram block or blocks. The methods and processes set forth herein need not, in some embodiments, be performed in the exact sequence as shown and likewise various blocks may be performed in parallel rather than in sequence. Accordingly, the elements of methods and processes are referred to herein as “blocks” rather than “steps.”

These computer program instructions may also be stored in a computer-readable memory that can direct a computer or other programmable data processing apparatus to function in a particular manner, such that the instructions stored in the computer-readable memory produce an article of manufacture including instructions, which implement the function/act specified in the flowchart and/or block diagram block or blocks.

The computer program instructions may also be loaded onto a computer or other programmable data processing apparatus that may be on or off-premises, or may be accessed via cloud in any of a software as a service (SaaS), platform as a service (PaaS), or infrastructure as a service (IaaS) architecture so as to cause a series of operational blocks to be performed on the computer or other programmable apparatus to produce a computer implemented process such that the instructions, which execute on the computer or other programmable apparatus provide blocks for implementing the functions/acts specified in the flowchart and/or block diagram block or blocks. It is contemplated that any part of any aspect or embodiment discussed in this specification can be implemented or combined with any part of any other aspect or embodiment discussed in this specification.

Further advantages and features consistent with this disclosure will be set forth in the following detailed description, with reference to the drawings.

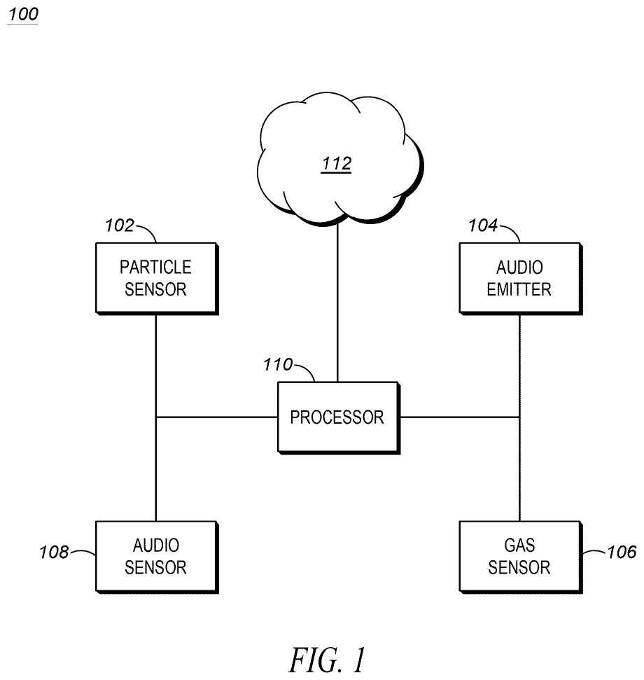

FIG. 1 depicts a block diagram 100 of an example air sensor for air pollutant detection in a location, in accordance with some examples. The example air sensor may comprise multiple components including a particle sensor 102, an audio emitter 104, a gas sensor 106, an audio sensor 108, and a processor 110 which may be connected to a cloud based computer/electronic device communication network. The particle sensor 102 may be configured to sense air particles in a particular input sample. The particle sensor 102 can determine and detect the presence of such air particles via suitable techniques such as light scattering (upon light colliding with particles), laser or electrostatic sensing, and/or particle concentration measurement. For example, the particle sensor 102 can provide a count for air particles of a specified size or sizes such as PM2.5 or PM10 in units of micrometers. That is, the particle sensor 102 may generate a count of specified small solid particles floating in the input sample, such as dust, smoke, or other particles.

The audio emitter 104 can emit audio for the example air sensor, such as for user voice control, user interactions, outputting an alert that an air pollutant of interest has been detected, and/or the like. In other words, the audio emitter 104 can function as a speaker and convert electrical signals to audible sound waves of a desired frequency range. The gas sensor 106 can detect particular gases in the input sample such as volatile organic compounds (VOC) that result from burning tobacco, for example. The gas sensor 106 can achieve this by generating an electric signal from a known chemical reaction to the desired gas being detected or via another suitable detection method. Additionally or alternatively, the gas sensor 106 can include a nicotine sensor component that detects the presence of nicotine.

The audio sensor 108 may be configured to sense audio data that corresponds to the location. The audio sensor 108 can perform signal processing for various audio analytics, noise level detection, sound pattern recognition, and/or the like for detected sound waves. For example, the audio sensor 108 can sense sounds, spoken keywords/phrases, and other audible indicia that can tend to indicate whether an initial air pollutant detection is accurate or inaccurate. The audio information from the audio sensor 108 may constitute audio contextual information that improves the accuracy and speed of air pollutant detection by the air sensor. The processor 110 may be in operative communication with the particle sensor 102, gas sensor 106, and audio sensor 108. In addition, the processor 110 may interoperate with other sensors, which could be components of the air sensor or be positioned remotely as independent sensors from the air sensor.

Other sensors may include motion sensors, thermal sensors, or other suitable sensors in electronic communication with the processor 110. For example, the thermal sensors can sense the presence of heat in order to provide an indication of a lighter spark (e.g., flame from cigarette lighter), match strike, or other heat signature suggestive of vaping or other undesirable air pollutant. For example, the motion sensors can sense the presence of a person to improve air pollutant detection since loitering may be suggestive of vaping while a detected absence of people in a room suggests it is impossible that vaping is occurring in that room. As such, the processor 110 may receive contextual cues (e.g., audio contextual cues) from the audio sensor 108 and other sensors as sensed data points to be analyzed in combination with direct air pollutant detection via the particle sensor 102 and the gas sensor 106.

For example, the processor 110 may extract particular audio keywords from audio samples captured by the audio sensor 108. The keywords may form a particular audible phrase that tends to suggest vaping is occurring. As an example, a spoken phrase such as “give me a hit” or words such as “e-cigarette” or “vape pen” can be identified by the audio sensor 108 and increase a vape detection score. In general, the sensed information described herein that tends to suggest or corroborate the occurrence of prohibited vaping or some other unauthorized air pollutant can be added to an initial air pollutant detection score which is compared to a detection threshold.

Accordingly, the vape or air pollutant detection algorithm of the present disclosure has improved accuracy, reduced false positives, and reduced time necessary to determine a detection. As described herein, each type of contextual cue or information can have a corresponding classification (e.g., contextual classification, audio classification) which can be used to determine a corresponding detection threshold or score. As an example, the audio detected by the audio sensor 108 can be classified into any number of suitable categories, such as lighter/match strike, cough, aerosol spray, keyphrases, etc. audio classifications. Similarly, other types of contextual information can be classified into various types of categories such as presence of people, sudden movements, etc. presence/motion classifications.

Each of the types of contextual information can independently change a current detection score (e.g., add to the current detection score) or a current detection threshold (e.g., increase or decrease threshold or change/set an expiration time for the threshold) based on the identity of the type of contextual information. Furthermore, different weights or detection scaling factors can be applied as desired according to the associated type of audio/contextual information. In general, these audio/contextual based adjustments to an air pollutant detection algorithm advantageously improve accuracy and efficiency.

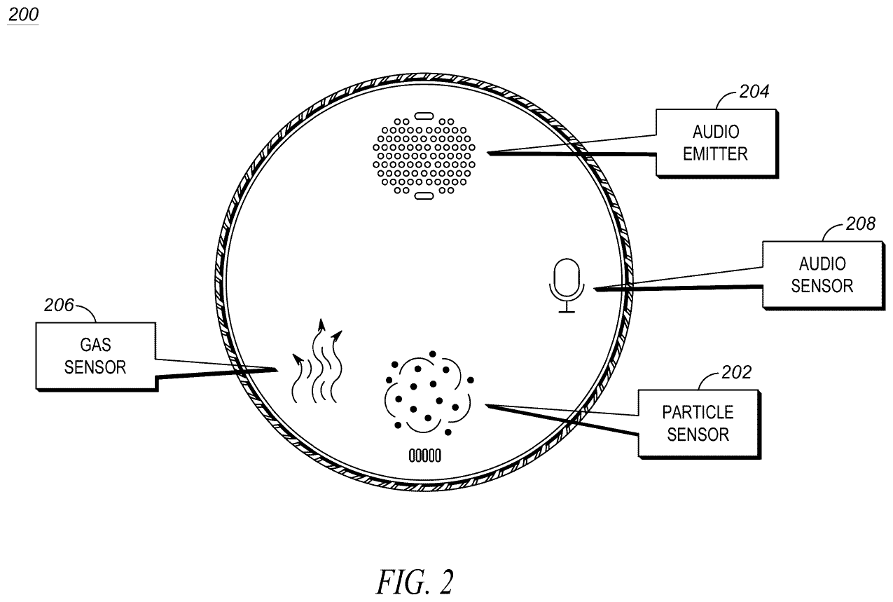

FIG. 2 depicts a top view 200 of an example air sensor and corresponding components, in accordance with some examples. The example air sensor can be configured to detect air pollutant(s) of interest in a particular location. The components may be similar or the same as those described in reference to FIG. 1, including a particle sensor 202, an audio emitter 204, a gas sensor 206, and an audio sensor 208, for example. The sensor components of the example air sensor may indicate contextual and audio information, such as audio detected events of interest (striking of a match, coughing, aerosol spraying, window closing, door opening, etc.), user detection (e.g., presence of person in room, loitering, etc.), flame or heat sources (e.g., high temperature from cigarette lighter), audio keyphrases (e.g., specific phrases such as “give me a hit”). As an example, according to the top view 200, the air sensor may comprise a circular housing with a microphone array of the audio emitter 204, the audio sensor 208 for audio analytics, and the particle sensor 202 and/or the gas sensor 206 for performing air pollutant detection.

Each of the particle sensor 202, the audio emitter 204, the gas sensor 206, and the audio sensor 208 may be located within a suitable place within the volume of the circular housing (or other type of housing configuration). For example, the particle sensor 202 can be located at the bottom of the air sensor device. The air sensor may be powered by a battery powered or plugged in power system. Other sensors or sensing components that are not shown in FIG. 2 may be part of the air sensor, such as an accelerometer, position sensor, motion sensor, thermal sensor, and/or the like. In addition, a processor and an onboard memory can be part of the air sensor. The processor and memory may be operatively connected with the sensor to execute instructions for performing of operations of a suitable air pollutant detection algorithm. As described herein, the air sensor may perform an improved algorithm for such detection, such as vaping detection based on audio and contextual cues acquired from the audio sensor 208 and contextual sensors (e.g., motion and thermal sensors), which can be onboard or remote from the air sensor.

As an example, the pollutant detection algorithm may involve checking audio and other contextual sensed data at a predetermined temporal cadence (e.g., every ten seconds or other time period) to increase or decrease a detection threshold for a specified amount of time depending on what audio and contextual cues are implicated. Additionally or alternatively, for a given clock/timer cycle, various contextual factors are assessed and analyzed for each cycle and used to adjust (e.g., add value) a current detection score for each type of factor according to a threshold counter and/or threshold test. Depending on the type of contextual or audio factor, the current detection score or threshold can be changed differently. For example, if the contextual factors include the audio sensor 208 sensing audible coughing as well as sniffling, this could increase or decrease the current detection threshold for a corresponding predetermined amount of time. Specifically, coughing can tend to suggest vape activity (e.g., smoking THC from a vape pen), so a detection of coughing by the audio sensor 208 may cause the vape detection threshold to be decreased or may add to the current vape detection score. This change in threshold or score may be associated with a countdown timer to reflect a certain time period for which the audio detection is associated with increased likelihood of vape detection.

Conversely, sniffing may not suggest any vaping activity or pollutant of interest, so a detection of sniffling by the audio sensor 208 may cause the detection threshold to be increased. This means vaping may be less likely to be detected according to the sniffling audio contextual cue, which is in contrast to the coughing scenario where vaping is more likely to be detected. As such, one example improved pollutant detection algorithm according to the present disclosure advantageously involves using different contextual factors to iteratively reduce detection thresholds by a specific value that differs depending on the type of contextual or audio factor. Furthermore, the factor based reduction in thresholds can be applied for a set period of time and/or according to a clock cycle. The associated severity of each contextual factor can vary depending on location where detection is occurring (e.g., single use bathrooms versus larger bathrooms). That is, the severity can be used as a criterion for how much a particular class of contextual or audio cue should independently adjust the corresponding detection threshold. As an example, an offender can spray deodorant to mask the smell of their vaping activity after entering the bathroom.

Conventionally, this might confuse or sufficiently reduce the sensitivity of the air sensor. However, the air sensor of the present disclosure advantageously can leverage contextual clues such as the offender of the example coughing a few times and an additional offender entering the bathroom and saying “give me a hit.” The coughing and audio keyword/keyphrase of interest can both cause the applicable detection threshold to be changed such that a previously undetected vaping pollutant activity can now be detected by the disclosed improved air sensor. As described herein, each adjustment from an audio or contextual cue can expire; that is, the adjustment can be applied for a predetermined amount of time, such as thirty seconds. As an example, for certain low motion sensors (e.g., passive infrared sensor) having intermittent detection, additional detections can cause the contextual cue based threshold adjustment to be applied.

Specifically, if a person is detected entering a room, a first motion detection could start a countdown timer for 30 seconds while any additional detections would reset the counter to 30 seconds. That is, additional instances of contextual or audio cues (which do not have to be the same type of contextual information which initially triggered the detection threshold adjustment) can reset or increase the predetermined amount of time (e.g., functioning as a time to live or expiration time). This means that a timely combination of detections of relevant contextual or audio information can cause the air sensor to remain in an “active” detection mode in which the contextual detections make it more likely that an accurate air pollutant detection can be made. The temporal threshold at which the adjustment is applied can be thirty seconds or any other suitable time. In general, one purpose of the temporal threshold is to ensure that a timely combination contextual cues with pollutant or vape detection is determined. For example, extending the effect of the cue on the threshold can improve detection for offenders that enter and leave quickly since it could take up to 30 s for a vape plume to fully travel to the air sensor.

As another example, there may be a delay between the detection of an audio or contextual event and the pollutant detection event. For example, two people may enter and one may say ‘give me a hit’, but it may take some time for them to start smoking in the location. Certain audio sounds or keyword/keyphrases may only occur once during a particular detection event and a mid-sentence pause may be indicative of an inhale event. Accordingly, applying the contextual or audio info triggered threshold change for a countdown timer or specific period of time and allowing that time period to reset can be beneficial for effectively implementing the improved vape detection algorithms of the present disclosure. Furthermore, some instances of contextual information may be suggestive of the pollutant of interest while other instances are not, so it is beneficial to have the flexibility of adjusting the detection threshold up or down. For example, one type of cough could indicate the coughing person is sick while another type of cough (e.g., cadence of cough) might indicate vaping activity such as smoking THC of a vape pen.

As described herein, the contextual enhanced pollutant detection algorithms may use or incorporate of a number of mathematical constructs such as numerical mean or averages of the pollutant detection and/or contextual data. The contextual and audio information pollutant detection algorithms of the present disclosure may or may not use a countdown timer(s) and/or detection score(s). The detection score(s) can have multiple components for the different types of audio and other contextual information being considered. The detection score(s) may represent a confidence level of the pollutant detection. The different types of detection audio and contextual information being detected can each increase or decrease the confidence in the accuracy of the air sensor's pollutant detection result. Some types of contextual information can render a positive detection result as effectively impossible; for example, if the motion sensor does not sense the presence of a person in the room, it is almost impossible that vaping activity can be correctly detected.

Depending on the contextual meaning of each type of audio and other contextual information, a different weighting factor can be applied for the pollutant detection algorithm being employed by the air sensor. In general, the disclosed improved pollutant/vape detection system and method advantageously can use any of several different mathematical approaches to combining analysis of the pollutant detection data and contextual data for quickly increasing detection accuracy and reducing false positives.

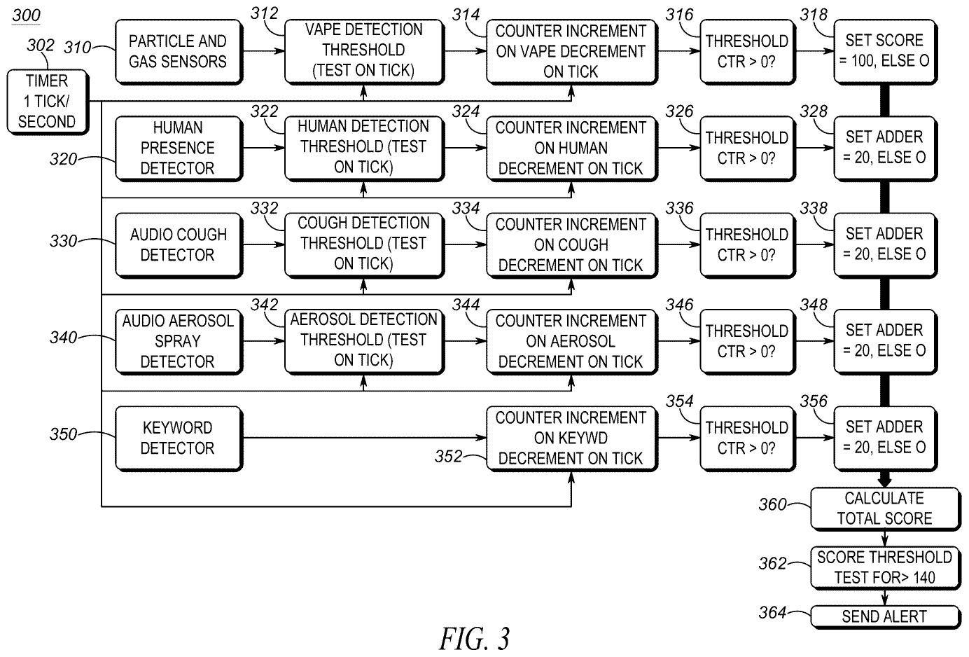

FIG. 3 illustrates a workflow 300 of a method for context enhanced air pollutant detection, in accordance with some examples. The workflow 300 depicts an example algorithm for improved pollutant detection (e.g., vaping detection) based on audio and other contextual information. As shown in FIG. 3, such information can be detected and/or monitored by multiple different types of sensors or sensing components, such as particle and gas sensing component 310, human presence detector 320, audio cough detector 330, audio aerosol spray detector 340, and keyword detector 350. The example algorithm can operate based on a clock, such as a timer that moves at one tick per second, although other suitable time cadences are also implementable. In other words, when each tick of the timer arrives, a test of each of the sensors of the air sensor disclosed herein is triggered.

Test on tick process blocks are shown in FIG. 3. For example, at step 312, if vape activity is detected based on a vape detection threshold, then a counter will be incremented by a set value. Different increment values can be applied depending on the vape activity; for example, a large counter can be applied for a larger detected vape plume. Alternatively, any detection by the particle and gas sensing component 310 that exceeds the vape detection threshold can increment/increase the vape counter by a fixed amount. Conversely, at step 314, for each tick of the timer, the vape counter can decrement by a value (e.g., fixed or unfixed). This process may iteratively repeat for each of the ticks on the timer, which could be a 30 second timer or some other suitable amount of time as appropriate.

As discussed above, the length of the timer enables pollutant detection to be considered in conjunction with relevant audio and other contextual information, which advantageously can improve detection accuracy and reduce detection false positives. Once the timer reaches zero, the workflow 300 proceeds to step 316 where a processor of the air sensor checks whether the threshold vape counter exceeds zero. If the counter is positive, then a current detection score is set at 100 at step 318. If the counter is nonpositive, then the current detection score is set at 0 at step 318. In this way, a base detection score for the air pollutant detection can be determined. Alternatively, the current detection score can be more granular such that different counter values could correspond to different scores such as 50, 70, or the like. Also, it should be noted that the 100 and 0 detection scores are merely representative examples and the detection algorithm is not limited to such values. Additionally, the detection score does not have to be set to a fixed amount for when the vape detection threshold is met. It can be a variable amount correlated with a confidence level of vape detection by the particle and gas sensing component 310.

As described herein, the air sensor of the present disclosure performs an improved pollutant/vape detection algorithm based on incorporating audio and other contextual information to adjust detection scores and/or thresholds. To that end, the human presence detector 320, audio cough detector 330, audio aerosol spray detector 340 are used in the workflow 300 to set detection thresholds that decay over time upon detection of the corresponding type of contextual information. At steps 322, 332, 342, the human detection threshold, cough detection threshold, and aerosol detection threshold are tested on each tick of the timer such that human presence, coughs, and aerosol sprays are assessed in a location (e.g., bathroom in a school) that the air sensor is monitoring. If any of the human presence, coughs, and aerosol sprays contextual information are detected as being present by the corresponding human presence detector 320, audio cough detector 330, audio aerosol spray detector 340, then the corresponding counter is incremented at steps 324, 334, 344 similarly to step 314. That is, on each tick, at steps 324, 334, 344 the respective detection threshold counters are decremented for each elapsed tick of time and incremented for each positive audio/contextual detection.

Similarly, the keyword detector 350 may monitor specific keywords or keyphrases such as “give me a hit” via the audio sensor 208. When particular keywords of interest that tend to suggest vaping activity is occurring are detected, then at step 352, the keyword counter is incremented. As each tick elapses, the keyword counter is decremented similarly to the other counters depicted in the workflow 300. It should be noted that the audio and contextual cue detectors shown in FIG. 3 are merely illustrative examples and other types of contextual information as described herein can also be used to enhance the effectiveness of pollutant detection by the air sensor. As shown in FIG. 3, for each tick of the timer, all the steps of the workflow 300 are performed. In this way, at each tick, a new calculated total or aggregate detection score is calculated at step 360.

The aggregate detection score can be based on the adjusted detection score which comprises the vape/pollutant detection score component and each type of detection score for the respective type of contextual information. At each tick, the current calculated aggregate detection score can be compared to a detection threshold such as a fixed aggregate detection threshold of 140 at step 362 as shown in FIG. 3. If the total aggregate score exceeds the detection threshold test value of 140 then an alert is generated and sent at step 364 to indicate that vape activity has occurred and/or the pollutant of interest is determined to be present in the location. In this way, for each iteration or tick of the workflow 300, the current detection score can be changed such as by adding different quantities to the current detection score (e.g., vape detection score) for each of the different types of contextual data which have positive threshold counter value values. In general, the current detection score can be changed by a quantity corresponding to the respective type of audio or other contextual data.

For example, audio data such as a cough compared to an audio keyphrase can change the current detection score by different quantities depending on how much the type of audio data makes it more likely that vape detection is actually correct. Similarly, the current detection score can be changed by a first quantity according to a type of contextual motion data and changed by a different second quantity according to the type of contextual thermal data. However, if desired, the quantity that is added to or used to change the current detection score can be the same for two or more different types of contextual data. In the example workflow 300 shown, the detection threshold value of 140 generally can be triggered by the base vape detection and two of the contextual adder detection scores. That is, an accurate detection of vaping or other pollutant activity can be confirmed by a positive detection by the particle and gas sensing component 310 in combination with two of the audio or other contextual cues being detected and having positive respective threshold counter values.

Accordingly, at steps 326, 336, 346, 354, each of the different types of threshold counters are tested at each tick of the timer to check whether they are above zero or not. At steps 328, 338, 348, 356, the adder is set at 20 if the respective counters are above zero, or else set at 0 if counter not above zero. It should be noted that although the adders in FIG. 3 are set at a fixed value of 20, other different values can be used, which can vary dynamically or all be the same constant value, depending on what type of contextual weighting factors are desired to be applied. In this connection, the vape detection score is set at 100 for a positive detection by the particle and gas sensing component 310 because any accurate detection of vaping activity requires a base detection of vape or other pollutants of interest by the air sensor. As described herein, other numerical construction and implementations are possible to implement the improved vape detection algorithm of the present disclosure. In general, the use of audio and other contextual cues in isolation or in combination such as detection of motion at a bathroom entrance, detection of lighter spark or match strike, detection of multiple people combined with wake word phrases such as “give me a hit”, detection of involuntary coughing, and identification of spray aerosol can as a masking attempt may be used to improve the accuracy, speed, and effectiveness of the vape detection algorithm.

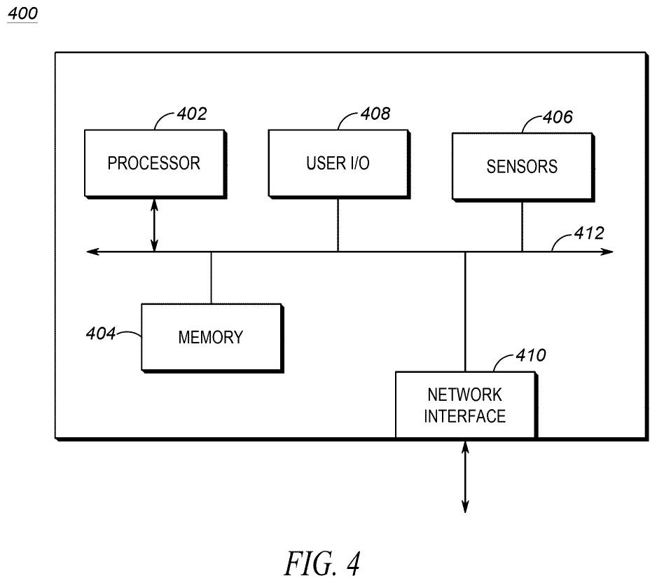

FIG. 4 illustrates a block diagram of an example electronic device 400 such as a computer device, in accordance with some examples. In some embodiments, the computer device 400 may be an air pollutant device or sensor, or a network device, or other equipment used in a network environment. The computer device 400 may include a physical device and/or a virtual device, such as a server running one or more virtual network functions (VNFs) of a network. The computer device 400 may be able to sense the presence of air pollutants of interest in a particular input sample. This air pollutant detection can be based on particle sensing and gas sensing components of the computer device 400. Moreover, the computer device 400 can perform improved air pollutant detection based on audio information and other contextual cues determined/received by contextual sensing components which can be on device or remotely located from the computer device 400. The contextual information comprise analytics used to increase speed and accuracy of pollutant detection by the computer device 400, as described herein.

In various examples, the computer device 400 may be or include a processor, a specialized computer, a personal or laptop computer (PC), a tablet PC, a mobile telephone, a smartphone, a network router, switch or bridge, a circuit such as an application specific integrated circuit (ASIC) or field programmable gate array (FPGA), or any machine capable of executing instructions (sequential or otherwise) that specify actions to be taken by that machine. The computer device 400 could be a remote computing device that is in operative communication with sensors (e.g., audio sensor, presence/motion sensor, thermal sensor, etc.). In some embodiments, the computer device 400 may be an internet-of-things (IoT) or a narrowband IoT (NB-IoT) device or other device embedded within other, non-communication-based devices such as appliances or vehicles. The computer device 400 may render a user interface for air pollutant detection, such as vape detection at a school environment or other controlled access or regulated environment.

The computer device 400 may include various components connected by a bus 412. The computer device 400 may include a hardware processor 402 such as one or more central processing units (CPUs) or other processing circuitry able to provide any of the functionality described herein when running instructions. The processor 402 may be connected to a memory 404 which may include a non-transitory machine-readable medium on which is stored one or more sets of instructions for air pollutant detection, for example. The memory 404 may include one or more of static or dynamic storage, or removable or non-removable storage, for example. A machine-readable medium may include any medium that is capable of storing, encoding, or carrying instructions for execution by the processor 402, such as solid-state memories, magnetic media, and optical media. The machine-readable medium may include, for example, Electrically Programmable Read-Only Memory (EPROM), Random Access Memory (RAM), or flash memory.

The instructions may enable the computer device 400 to operate in any manner thus programmed, such as the functionality described specifically herein, when the processor 402 executes the instructions. The machine-readable medium may be stored as a single medium or in multiple media, in a centralized or distributed manner. In some embodiments, instructions may further be transmitted or received over a communications network via a network interface 410 utilizing any one of a number of transfer protocols (e.g., frame relay, internet protocol (IP), transmission control protocol (TCP), user datagram protocol (UDP), hypertext transfer protocol (HTTP), etc.).

The network interface 410 may thus enable the computer device 400 to communicate data and control information (e.g., security information) with other devices via wired or wireless communication. The network interface 410 may include electronic components such as a transceiver that enables serial or parallel communication. The wireless connections may use one or more protocols, including Institute of Electrical and Electronics Engineers (IEEE) Wi-Fi 802.11, Long Term Evolution (LTE)/4G, 5G, Universal Mobile Telecommunications System (UMTS), or peer-to-peer (P2P), for example, or short-range protocols such as Bluetooth, Zigbee, or near field communication (NFC). Wireless communication may occur in one or more bands, such as the 800-900 MHz range, 1.8-1.9 GHz range, 2.3-2.4 GHz range, 60 GHz range, and others, including infrared (IR) communications. Example communication networks to which computer device 400 may be connected via network interface 410 may include a local area network (LAN), a wide area network (WAN), a packet data network (e.g., the Internet), mobile telephone networks (e.g., cellular networks), and wireless data networks. The computer device 400 may be connected to the networks via one or more wired connectors, such as a universal serial bus (USB), and/or one or more wireless connections, and physical jacks (e.g., Ethernet, coaxial, or phone jacks) or antennas.

The computer device 400 may further include one or more sensors 406, such as one or more of an image sensor, a motion sensor, an audio sensor, a global positioning system (GPS) sensor, a thermometer or thermal sensor, a magnetometer, a barometer, a pedometer, a proximity sensor, a door sensor, or an ambient light sensor, among others. The sensors 406 may include some, all, or none of one or more of the types of sensors above (although other types of sensors may also be present), as well as one or more sensors of each type. The sensors 406 may be used in conjunction with the computer device 400 to indicate contextual and audio information, such as audio detected events of interest (striking of a match, coughing, aerosol spraying, window closing, door opening, etc.), user detection (e.g., presence of person in room, loitering, etc.), flame or heat sources (e.g., high temperature from cigarette lighter), audio keyphrases (e.g., specific phrases such as “give me a hit”). In this way, air pollutant detection may be improved. The detected pollutants of interest as well as other sensed information can be retrieved, analyzed, and otherwise manipulated by users via one or more user input/output (I/O) devices 408. The user I/O devices 408 may include one or more of a display (e.g., a touch screen display of a mobile computing device), a camera, a speaker, a keyboard, a microphone, a mouse (or other navigation device), or a fingerprint scanner, among others. The user I/O devices 408 may include some, all, or none of one or more of the types of I/O devices above (although other types of I/O devices may also be present), as well as one or more I/O devices of each type.

The computer device 400 may include different specific elements depending on the particular device. For example, although not shown, in some embodiments, computer device 400 may include a front end that incorporates a millimeter and sub-millimeter wave radio front end module integrated circuit (RFIC) connected to the same or different antennae. The RFICs may include processing circuitry that implements processing of signals for the desired protocol (e.g., medium access control (MAC), radio link control (RLC), packet data convergence protocol (PDCP), radio resource control (RRC) and non-access stratum (NAS) functionality) using one or more processing cores to execute instructions and one or more memory structures to store program and data information. The RFICs may further include digital baseband circuitry, which may implement physical layer functionality (such as hybrid automatic repeat request (HARQ) functionality and encoding/decoding, among others), transmit and receive circuitry (which may contain digital-to-analog and analog-to-digital converters, up/down frequency conversion circuitry, filters, and amplifiers, among others), and RF circuitry with one or more parallel RF chains for transmit/receive functionality (which may contain filters, amplifiers, phase shifters, up/down frequency conversion circuitry, and power combining and dividing circuitry, among others), as well as control circuitry to control the other RFIC circuitry.

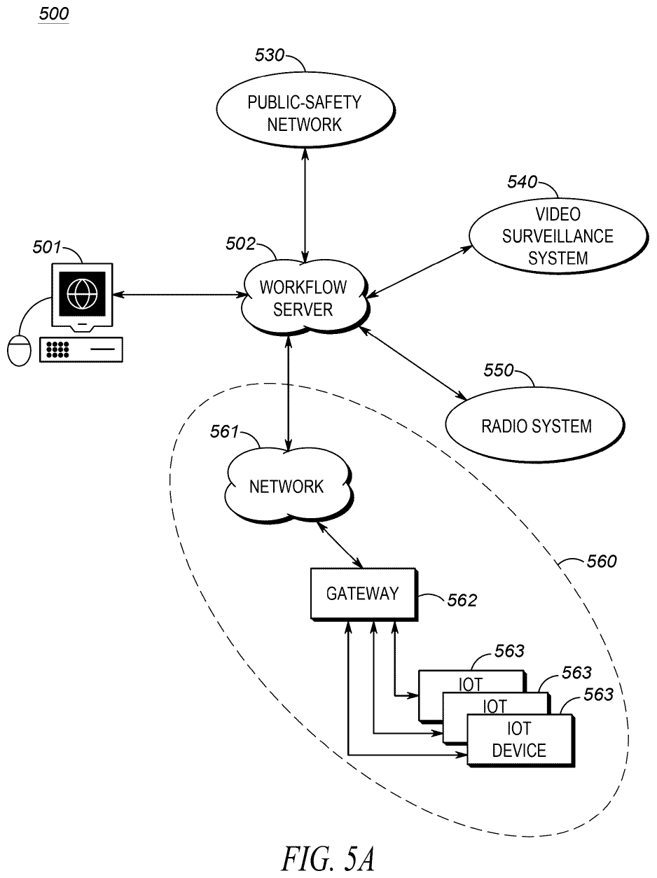

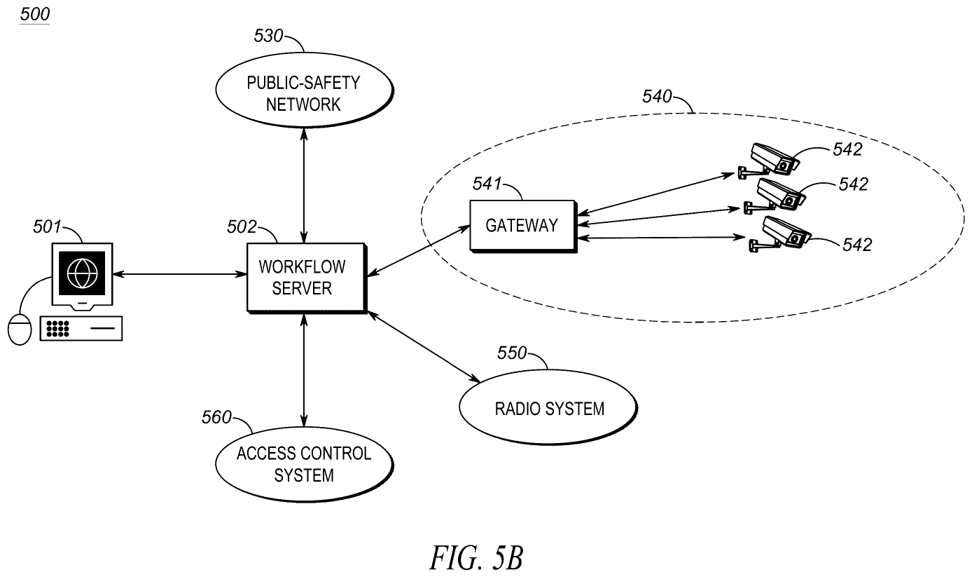

FIGS. 5A-5B each illustrate a security ecosystem 500 comprising a plurality of electronic devices, in accordance with some examples. The security ecosystem 500 can be capable of monitoring an environment (e.g., substance controlled environment such as a school) for air pollutant detection (e.g., such as to prevent students from smoking and vaping). FIG. 5A illustrates the security ecosystem 500 capable of configuring and automating workflows across multiple systems. As shown, the security ecosystem 500 comprises a public-safety network 530, a video surveillance system 540, a private radio system 550, and an access control system 560. The workflow server 502 is coupled to each system 530, 540, 550, and 560. The workstation 501 is shown coupled to the workflow server 502, and is utilized to configure server 502 with workflows created by a user. It should be noted that although the components in FIG. 5 are shown geographically separated, these components can exist within a same geographic area, such as, but not limited to a building, a school, a hospital, an airport, a sporting event, concert, marathon, a stadium, etc. It should also be noted that although only the networks and systems 530-560 are shown in FIG. 5A, one of ordinary skill in the art will recognize that many more networks and systems may be included in ecosystem 500.

The workstation 501 is preferably a computer configured to execute dispatch and incident management software. As will be discussed in more detail below, the workstation 501 is configured to present a user with a plurality of triggers capable of being detected by the network and systems 530-560 as well as present the user with a plurality of actions capable of being executed by the network and systems 530-560. The user will be able to create workflows and upload these workflows to the workflow server 502 based on the presented triggers and actions. For example, an example trigger is an emergency trigger sent from or to the workstation 501. This can cause a message to be sent to administrative personnel to respond to a detected incident such as a dangerous air pollutant being present, vaping incident, health incident, and/or the like.

The workflow server 502 is preferably a server running a command center software and platform. The workflow server 502 is configured to receive workflows created by the workstation 501 and implement the workflows. Particularly, the workflows are implemented by analyzing events detected by the network and systems 530-560 and executing appropriate triggers. For example, assume a user creates a workflow on the workstation 501 that has a trigger comprising the surveillance system 540 detecting a loitering event, and has an action comprising notifying radios within the public-safety network 530. The loitering event could be indicative of a vaping event, for example. Furthermore, the video surveillance system 540 can comprise an air sensor as described herein. The air sensor can be configured to detect vape or other pollutants of interest in an input air sample and can be used in conjunction or independently of video surveillance to trigger and/or implement workflows. Also, the air sensor could be located independently from the video surveillance system 540 as a separate sensor component of the security ecosystem 500. When this workflow is uploaded to the workflow server 502, the workflow server 502 will notify the radios of any loitering event detected by the surveillance system 540 in communication with the workflow server 502. As an example, the workflow server 502 may be configured to decode encoded data, such as data encoded on visual or color coded signals broadcast or propagated by/within a network of electronic devices.

The public-safety network 530 is configured to detect various triggers and report the detected triggers to the workflow server 502. The public-safety network 530 is also configured to receive action commands from the workflow server 502 and execute the actions. In one embodiment of the present invention, the public-safety network 530 comprises includes typical radio-access network (RAN) elements such as base stations, base station controllers (BSCs), routers, switches, and the like, arranged, connected, and programmed to provide wireless service to user equipment, report detected events, and execute actions received from the workflow server 502.

The video surveillance system 540 is configured to detect various triggers and report the detected triggers to the workflow server 502. The public-safety network 530 is also configured to receive action commands from workflow server 502 and execute the actions. For example, the video surveillance system 540 comprises a plurality of video cameras that may be configured to automatically change their field of views over time. The video surveillance system 540 is configured with a recognition engine/video analysis engine (VAE) that comprises a software engine that analyzes any video captured by the cameras. Using the VAE, the video surveillance system 540 is capable of “watching” video to detect any triggers and report the detected triggers to workflow server 502. Similarly, the video surveillance system 540 can execute action commands received from the workflow server 502. As described herein, the triggers and action commands can be for managing the detected presence of dangerous air pollutants or other prohibited substances in the air or activities of interest by suspected offenders.

The radio system 550 preferably comprises a private enterprise radio system that is configured to detect various triggers and report the detected triggers to the workflow server 502. The radio system 550 is also configured to receive action commands from workflow server 502 and execute the actions. For example, the radio system 550 may comprise a MOTOTRBO™ communication system having radio devices that operate in the CBRS spectrum and combines broadband data with voice communications.

The access control system 560 may comprise an IoT network. The IoT system 560 serves to connect every-day devices to the Internet. Devices such as cars, kitchen appliances, medical devices, sensors, doors, windows, HVAC systems, drones, . . . , etc. can all be connected through the IoT. Basically, anything that can be powered can be connected to the internet to control its functionality. The system 560 allows objects to be sensed or controlled remotely across existing network infrastructure. For example, the access control system 560 may be configured to provide access control to various doors and windows. With this in mind, the access control system 560 is configured to detect various triggers (e.g., door opened/closed) and report the detected triggers to workflow server 502. As an example, the access control system 560 is configured to control access to a location, such as a restricted area of a school. The access control system 560 is also configured to receive action commands from the workflow server 502 and execute the action received from the workflow server 502. The action commands may take the form of instructions to lock, open, and/or close a door or window, or to allow a participant to enter.

As is evident, the above security ecosystem 500 allows an administrator using the workstation 501 to create rule-based, automated workflows between technologies to enhance efficiency, and improve response times, effectiveness, and overall safety. The above ecosystem 500 has the capability to detect triggers across a number of devices within the network and systems 530-560 quickly take actions by automatically executing the proper procedure (i.e., executing the appropriate action once a trigger is detected).

FIG. 5B illustrates a security ecosystem capable of configuring and automating workflows. In particular, FIG. 5B shows the security ecosystem 500 with an expanded view of an access control system 560. As shown, the access control system 560 comprises a plurality of IoT devices 563 coupled to the gateway 562. Data passed from the workflow server 502 to the IoT devices 563 passes through the network 561, gateway 562 and ultimately to the IoT device 563. Conversely, data passed from the IoT devices 563 to the workflow server 502 passes through the gateway 562, network 561, and ultimately to the workflow server 502.

The IoT devices 563 preferably comprise devices that control objects, doors, windows, sensors, . . . , etc. As is known in the art, a particular communication protocol (IoT protocol) may be used for each IoT device. For example, various proprietary protocols such as DNP, Various IEC**** protocols (IEC 61850 etc . . . ), bacnet, EtherCat, CANOpen, Modbus/Modbus TCP, EtherNet/IP, PROFIBUS, PROFINET, DeviceNet, . . . , etc. can be used. Also a more generic protocol such as Coap, Mqtt, and RESTfull may also be used.

The gateway 562 preferably comprises an Avigilon™ Control Center running Avigilon's Access Control Management software. The gateway 562 is configured to run the necessary Application Program Interface (API) to provide communications between any IoT device 563 and the workflow server 502.

The network 561 preferably comprises one of many networks used to transmit data, such as but not limited to a network employing one of the following protocols: a Long Term Evolution (LTE) protocol, LTE-Advance protocol, or 5G protocol including multimedia broadcast multicast services (MBMS) or single site point-to-multipoint (SC-PTM) protocol over which an open mobile alliance (OMA) push to talk (PTT) over cellular protocol (OMA-PoC), a voice over IP (VoIP) protocol, an LTE Direct or LTE Device to Device protocol, or a PTT over IP (PoIP) protocol, a Wi-Fi protocol perhaps in accordance with an IEEE 802.11 standard (e.g., 802.11a, 802.11b, 802.11g) or a WiMAX protocol perhaps operating in accordance with an IEEE 802.16 standard.

As discussed herein, the security ecosystem 500 is capable of configuring and automating workflows. In particular, FIG. 5B shows the security ecosystem 500 with an expanded view of the video surveillance system 540. As shown, the video surveillance system 540 comprises a plurality of cameras 542 and gateway 541. The cameras 542 may be fixed or mobile, and may have pan/tilt/zoom (PTZ) capabilities to change their field of view. The cameras 542 may also comprise circuitry configured to serve as a video analysis engine (VAE) which comprises a software engine that analyzes analog and/or digital video. The engine is configured to “watch” video and detect pre-selected objects such as people, faces, vape pens, etc.. The software engine may also be configured to detect certain actions of individuals, such as fighting, loitering, lighting up, crimes being committed, . . . , etc. The VAE may contain any of several object/action detectors.

Each object/action detector “watches” the video for a particular type of object or action. Object and action detectors can be mixed and matched depending upon what is trying to be detected. For example, an automobile object detector may be utilized to detect automobiles, while a fire detector may be utilized to detect fires. The gateway 541 preferably comprises an Avigilon™ Control Center running Avigilon's Access Control Management software. The gateway 541 is configured to run the necessary Application Program Interface (API) to provide communications between any cameras 542 and the workflow server 502.

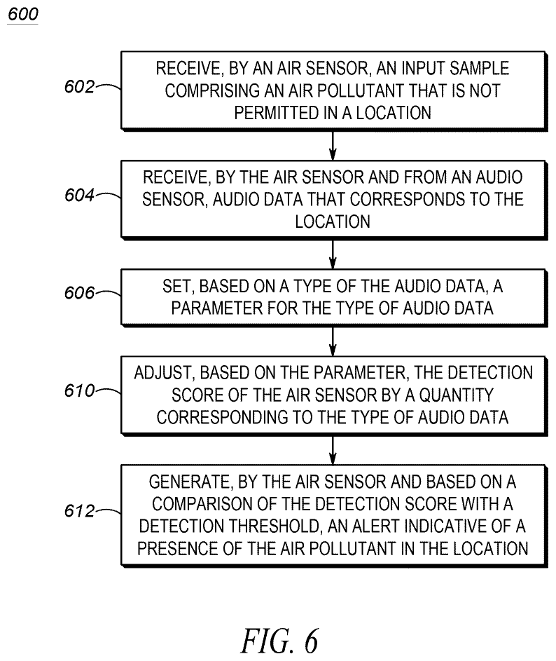

FIG. 6 illustrates an example flow diagram (e.g., process 600) for air pollutant detection in a particular location, according to certain aspects of the present disclosure. For explanatory purposes, the example process 600 is described herein with reference to one or more of the figures above. Further for explanatory purposes, the blocks of the example process 600 are described herein as occurring in serial, or linearly. However, multiple instances of the example process 600 may occur in parallel, overlapping in time, almost simultaneously, or in a different order from the order illustrated in the process 600. In addition, the blocks of the example process 600 need not be performed in the order shown and/or one or more of the blocks of the example process 600 need not be performed.

At step 602, an input sample comprising an air pollutant that is not permitted in a location may be received by an air sensor. The air sensor may comprise at least one of a particle sensor or a gas senor. The air sensor receives the input sample and is configured to sense that the air pollutant is present. As an example, the input sample corresponds to a detection score. As an example, receiving the input sample comprises determining a masking substance of the input sample comprises an aerosol spray or chemical substance that impedes detection of the air pollutant. The air pollutant can comprise nicotine particles. According to an aspect, the air sensor comprises or is part of a system comprising a motion sensor configured to sense movement at the location and an audio sensor configured to sense audio data audio data that corresponds to the location.

At step 604, audio data that corresponds to the location can be received by the air sensor from the audio sensor. As an example, receiving the audio data comprises determining at least one of: ignition of a fuel, spraying of an aerosol, detection of an audible phrase, or detection of coughing. A processor may be operatively in communication with the air sensor, the motion sensor, and the audio sensor, that, upon executing program instructions, determines a detection score for the input sample. At step 606, a parameter for the type of audio data is set based on a type of the audio data. According to an aspect, the processor may be configured to set the parameter for the type of audio data. For example, setting the parameter comprises determining an audio classification for the type of audio data. For example, setting the parameter comprises determining an audio detection threshold for the type of audio data being monitored.

At step 608, the detection score of the air sensor may be adjusted by a quantity corresponding to the type of audio data based on the parameter. For example, adjusting the detection score comprises adding a quantity to the detection score, wherein a value of the quantity is based on the type of audio data. For example, adjusting the detection score comprises adjusting the detection score for a predetermined period of time. According to an aspect, the processor may be configured to determine an aggregate detection score based on the adjusted detection score and motion data from the motion sensor.

At step 610, an alert indicative of a presence of the air pollutant in the location may be generated by the air sensor and based on a comparison of the detection score with a detection threshold. For example, generating the alert comprises generating an indication that vape activity has occurred and wherein the air sensor comprises a gas sensor configured to detect a presence of a nicotine based substance. According to an aspect, the processor can be configured to generate, via the air sensor and based on a comparison of the aggregate detection score with a detection threshold, an alert indicative of a presence of the air pollutant in the location. As an example, the processor can be configured to add a first quantity to the aggregate detection score, wherein the first quantity is based on the type of audio data. As an example, the processor can be configured to add a second quantity to the aggregate detection score, wherein the second quantity is based on the type of motion data.

According to an aspect, the process 600 comprises receiving, by the air sensor and from a motion sensor, motion data that corresponds to the location. According to an aspect, the process 600 comprises receiving, by the air sensor and from a thermal sensor, thermal data that corresponds to the location. According to an aspect, the process 600 comprises changing the detection score by a first quantity corresponding to a type of the motion data and by a second quantity corresponding to a type of the thermal data. According to an aspect, the process 600 comprises receiving, from a contextual sensor, contextual data corresponding to the location.

According to an aspect, the process 600 comprises setting, based on a type of the audio data, an audio parameter for the type of audio data. According to an aspect, the process 600 comprises setting, based on a type of the contextual data, a contextual parameter for the type of contextual data. For example, the processor is configured to determine an contextual classification for the type of contextual data. For example, the processor is configured to determine an motion detection threshold or thermal detection threshold for the type of contextual data being monitored. According to an aspect, the process 600 comprises adjusting, based on the audio parameter and the contextual parameter, a detection score of the air sensor by a quantity corresponding to the type of audio data and the type of contextual data. According to an aspect, the process 600 comprises adding a first quantity to the detection score, wherein the first quantity is based on the type of audio data. According to an aspect, the process 600 comprises adding a second quantity to the detection score, wherein the second quantity is based on the type of contextual data.

As should be apparent from this detailed description above, the operations and functions of electronic computing devices described herein are sufficiently complex as to require their implementation on a computer system, and cannot be performed, as a practical matter, in the human mind. Electronic computing devices such as set forth herein are understood as requiring and providing speed and accuracy and complexity management that are not obtainable by human mental steps, in addition to the inherently digital nature of such operations (e.g., a human mind cannot interface directly with RAM or other digital storage, cannot transmit or receive electronic messages, validate digital certificates, issue tokens, and the like).

In the foregoing specification, specific examples have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

A device or structure that is “configured” in a certain way is configured in at least that way, but may also be configured in ways that are not listed.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through intermediate elements or devices via an electrical element, electrical signal or a mechanical element depending on the particular context.

Also, it should be understood that the illustrated components, unless explicitly described to the contrary, may be combined or divided into separate software, firmware, and/or hardware. For example, instead of being located within and performed by a single electronic processor, logic and processing described herein may be distributed among multiple electronic processors. Similarly, one or more memory modules and communication channels or networks may be used even if embodiments described or illustrated herein have a single such device or element. Also, regardless of how they are combined or divided, hardware and software components may be located on the same computing device or may be distributed among multiple different devices. Accordingly, in this description and in the claims, if an apparatus, method, or system is claimed, for example, as including a controller, control unit, electronic processor, computing device, logic element, module, memory module, communication channel or network, or other element configured in a certain manner, for example, to perform multiple functions, the claim or claim element should be interpreted as meaning one or more of such elements where any one of the one or more elements is configured as claimed, for example, to make any one or more of the recited multiple functions, such that the one or more elements, as a set, perform the multiple functions collectively.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Any suitable computer-usable or computer readable medium may be utilized. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. In the context of this document, a computer-usable or computer-readable medium may be any medium that can contain, store, communicate, propagate, or transport the program for use by or in connection with the instruction execution system, apparatus, or device.

Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. For example, computer program code for carrying out operations of various example embodiments may be written in an object oriented programming language such as Java, Smalltalk, C++, Python, or the like. However, the computer program code for carrying out operations of various example embodiments may also be written in conventional procedural programming languages, such as the “C” programming language or similar programming languages. The program code may execute entirely on a computer, partly on the computer, as a stand-alone software package, partly on the computer and partly on a remote computer or server or entirely on the remote computer or server. In the latter scenario, the remote computer or server may be connected to the computer through a local area network (LAN) or a wide area network (WAN), or the connection may be made to an external computer (for example, through the Internet using an Internet Service Provider).

The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “one of”, without a more limiting modifier such as “only one of”, and when applied herein to two or more subsequently defined options such as “one of A and B” should be construed to mean an existence of any one of the options in the list alone (e.g., A alone or B alone) or any combination of two or more of the options in the list (e.g., A and B together). Similarly the terms “at least one of” and “one or more of”, without a more limiting modifier such as “only one of”, and when applied herein to two or more subsequently defined options such as “at least one of A or B”, or “one or more of A or B” should be construed to mean an existence of any one of the options in the list alone (e.g., A alone or B alone) or any combination of two or more of the options in the list (e.g., A and B together).

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method comprising:
receiving, by an air sensor, an input sample comprising an air pollutant that is not permitted in a location, wherein the input sample corresponds to a detection score;
receiving, by the air sensor and from an audio sensor, audio data that corresponds to the location;
setting, based on a type of the audio data, a parameter for the type of audio data;
adjusting, based on the parameter, the detection score of the air sensor by a quantity corresponding to the type of audio data;
generating, by the air sensor and based on a comparison of the detection score with a detection threshold, an alert indicative of a presence of the air pollutant in the location.

2. The method of claim 1, wherein receiving the input sample comprises determining a masking substance of the input sample comprises an aerosol spray or chemical substance that impedes detection of the air pollutant, wherein the air pollutant comprises nicotine particles and wherein the air sensor comprises at least one of a particle sensor or a gas sensor.

3. The method of claim 1, wherein receiving the audio data comprises determining at least one of: ignition of a fuel, spraying of an aerosol as a masking substance, detection of an audible phrase, or detection of coughing.

4. The method of claim 1, wherein setting the parameter comprises:
determining an audio classification for the type of audio data; and
determining, based on the audio classification, an audio detection threshold for the type of audio data being monitored.

5. The method of claim 1, wherein adjusting the detection score comprises adding a quantity to the detection score, wherein a value of the quantity is based on the type of audio data.

6. The method of claim 1, wherein adjusting the detection score comprises adjusting the detection score for a predetermined period of time.

7. The method of claim 1, further comprising:
receiving, by the air sensor and from a motion sensor, motion data that corresponds to the location;
receiving, by the air sensor and from a thermal sensor, thermal data that corresponds to the location; and
changing the detection score by a first quantity corresponding to a type of the motion data and by a second quantity corresponding to a type of the thermal data.

8. The method of claim 1, wherein generating the alert comprises generating an indication that vape activity has occurred and wherein the air sensor comprises a gas sensor configured to detect a presence of a nicotine based substance.

9. A system comprising:
an air sensor configured to sense that an air pollutant of interest is present, wherein the air sensor receives an input sample comprising the air pollutant that is not permitted in a location;
a motion sensor configured to sense movement at the location;
an audio sensor configured to sense audio data audio data that corresponds to the location; and
a processor operatively in communication with the air sensor, the motion sensor, and the audio sensor, that, upon executing program instructions, is configured to:
determine a detection score for the input sample;
set, based on a type of the audio data, a parameter for the type of audio data;
adjust, based on the parameter, the detection score of the air sensor by a quantity corresponding to the type of audio data;
determine an aggregate detection score based on the adjusted detection score and motion data from the motion sensor; and
generate, via the air sensor and based on a comparison of the aggregate detection score with a detection threshold, an alert indicative of a presence of the air pollutant in the location.

10. The system of claim 9, wherein the air sensor is configured to determine that a masking substance of the input sample comprises an aerosol spray or chemical substance that impedes detection of the air pollutant, wherein the air pollutant comprises nicotine particles and the air sensor comprise at least one of a particle sensor or a gas sensor.

11. The system of claim 9, wherein the processor is configured to set the parameter by being configured to:
determine an audio classification for the type of audio data; and
determine, based on the audio classification an audio detection threshold for the type of audio data being monitored.

12. The system of claim 9, wherein the processor is configured to determine the aggregate detection score by being configured to:
add a first quantity to the aggregate detection score, wherein the first quantity is based on the type of audio data; and
add a second quantity to the aggregate detection score, wherein the second quantity is based on the type of motion data.

13. The system of claim 9, wherein the processor is further configured to determine that the type of audio data comprises at least one of: ignition of a fuel, spraying of an aerosol as the masking substance, detection of an audible phrase, or detection of coughing.

14. The system of claim 9, wherein the processor is further configured to generate the alert by being configured to generate an indication that vape activity has occurred.

15. A air sensor device comprising:
a gas sensor configured to sense that an air pollutant of interest is present based on a received input sample comprising the air pollutant that is not permitted in a location;
a processor; and
a computer-readable storage medium having stored thereon program instructions that, when executed by the processor, cause the air sensor device to perform a set of operations comprising:
determining a detection score for the input sample;
receiving, from an audio sensor, audio data that corresponds to the location;
receiving, from a contextual sensor, contextual data corresponding to the location;
setting, based on a type of the audio data, an audio parameter for the type of audio data;
setting, based on a type of the contextual data, a contextual parameter for the type of contextual data;
adjusting, based on the audio parameter and the contextual parameter, a detection score of the air sensor by a quantity corresponding to the type of audio data and the type of contextual data;
generating, based on a comparison of the detection score with a detection threshold, an alert indicative of a presence of the air pollutant in the location.

16. The air sensor device of claim 15, wherein the gas sensor is configured to determine a masking substance comprises an aerosol spray or chemical substance that impedes detection of the air pollutant, wherein the air pollutant comprises nicotine particles.

17. The air sensor device of claim 15, wherein the set of operations comprising setting the contextual parameter comprise:
determining an contextual classification for the type of contextual data; and
determining, based on the contextual classification, an motion detection threshold or thermal detection threshold for the type of contextual data being monitored.

18. The air sensor device of claim 15, wherein the set of operations comprising adjusting the detection score comprise:
adding a first quantity to the detection score, wherein the first quantity is based on the type of audio data; and
adding a second quantity to the detection score, wherein the second quantity is based on the type of contextual data.

19. The air sensor device of claim 15, wherein the set of operations comprising generating the alert comprises generating an indication that vape activity has occurred.

20. The air sensor device of claim 15, wherein the set of operations further comprise determining that the type of audio data comprises at least one of: ignition of a fuel, spraying of an aerosol as a masking substance, detection of an audible phrase, or detection of coughing.
